(19) United States
(12) Patent Application Publication
(10) Pub. No.: US 2022/0157404 A1
(43) Pub. Date: May 19, 2022

(54) USING RELATIVES' INFORMATION TO DETERMINE GENETIC RISK FOR NON-MENDELIAN PHENOTYPES

(71) Applicant: THEMBA INC., SAN CARLOS, CA (US)

(72) Inventor: MATTHEW RABINOWITZ, SAN FRANCISCO, CA (US)

(73) Assignee: THEMBA INC., SAN CARLOS, CA (US)

(21) Appl. No.: 17/440,548

(22) PCT Filed: Mar. 19, 2020
(86) PCT No.: PCT/US2020/023633

§ 371 Date: Sep. 17, 2021

Related U.S. Application Data
(60) Provisional application No. 62/820,286, filed on Mar. 19, 2019.

(51) Int. Cl. G16B 40/20; G16B 20/20; G16B 25/10; G16H 50/30; G16H 10/60

(52) U.S. Cl. CPC G16B 40/20; G16B 20/20; G16H 10/60; G16H 50/30; G16B 25/10

(57) ABSTRACT

Provided are methods for outputting a non-Mendelian risk score, comprising: receiving from a first dataset (i) genotype data for a subject and (ii) genotype data and phenotype data for one or more blood relatives of a subject having a gene of interest; receiving from a second dataset genotype population data and phenotype population data, wherein the population comprises two or more blood relatives; training a model on the first and second datasets to determine a genetic risk in the subject associated with one or more non-Mendelian gene of interest; and outputting a phenotypic risk score for the subject. Also provided are systems and non-transitory machine-readable media for outputting a polygenic risk score for a subject.

### CROSS REFERENCE TO RELATED APPLICATIONS

This application claims the benefit of U.S. Provisional Application No. 62/820,286, filed on Mar. 19, 2019, which is incorporated herein by reference in their entirety.

### FIELD

Described are methods for determining genetic risk of non-Mendelian phenotypes using relatives' genetic information.

### BACKGROUND

For Mendelian genes, the probability of developing a phenotype is roughly 0 or 1, depending on whether or not the subject inherits 0, 1 or 2, versions of the mutated gene and whether the gene displays dominant or recessive inheritance. For Mendelian phenotypes, risk for a subject is established by analyzing the family tree and disease history of the subject's relatives in a well-defined manner. For non-Mendelian genes, the probability of a subject with a particular gene mutation developing a phenotype is not absolutely 0 or 1. In addition, non-Mendelian phenotypes are typically affected by multiple genes. The effect of multiple genes is typically captured in polygenic risk models, which tend to be inaccurate and use population-level data to calibrate the effect of each gene. There is a need in the art for more precise methods for determining whether a subject is it risk for a non-Mendelian phenotype, particularly methods that can incorporate family disease history.

### SUMMARY

Provided are methods for outputting a non-Mendelian phenotypic risk score that is made more accurate for each subject by using the disease or phenotype status of the subject's relatives. Some aspects comprise receiving from a first dataset (i) genotype data for a subject having one or more non-Mendelian genes of interest and (ii) genotype data and phenotype data for one or more blood relatives of the subject that have one or more of the non-Mendelian genes of interest. Some aspects comprise receiving from a second dataset genotype population data and phenotype population data, wherein the population comprises one or more sets of two or more blood relatives. Some aspects comprise training a model on the first and second datasets to determine a risk in the subject associated with one or more of the non-Mendelian genes of interest. Some aspects comprise outputting a phenotypic risk score for the subject.

In some aspects, the second dataset comprises genotype population data and phenotype population data for more than one set of two or more blood relatives.

In some aspects, the blood relative in the first dataset comprises one or more of the subject's mother, father, brother, sister, son, daughter, grandfather, grandmother, aunt, uncle, niece, nephew, and first cousin. In some aspects, the second dataset includes two or more subjects having the same blood relationship as the subjects in the first dataset.

In some aspects, one or more of the blood relatives is a male relative. In some aspects, one or more of the blood relatives is a female relative.

In some aspects, the first dataset includes data for more than one blood relative of the subject. In some aspects, one or more of the blood relatives is a male relative and one or more of the blood relatives is a female relative.

In some aspects, the gene of interest is a genetic variant of interest.

In some aspects, the first dataset and second dataset include data associated with the age of onset of the phenotype.

Also provided are systems comprising: a processor; a memory coupled to the processor to store instructions which, when executed by the processor, cause the processor to perform operations, the operations including: receiving from a first dataset (i) genotype data for a subject having one or more non-Mendelian genes of interest and (ii) genotype data and phenotype data for one or more blood relatives of the subject that have one or more of the genes of interest; receiving from a second dataset genotype population data and phenotype population data, wherein the population comprises one or more sets of two or more blood relatives; training a model on the first and second datasets to determine a risk in the subject associated with one or more of the non-Mendelian gene of interest, and outputting a phenotypic risk score for the subject.

Also provided are non-transitory machine-readable media having instructions stored therein which, when executed by a processor, cause the processor to perform operations, the operations comprising: receiving from a first dataset (i) genotype data for a subject having one or more non-Mendelian genes of interest and (ii) genotype data and phenotype data for one or more blood relatives of the subject that have one or more of the genes of interest; receiving from a second dataset genotype data and phenotype population data, wherein the population comprises one or more sets of two or more blood relatives; training a model on the first and second datasets to determine a risk in the subject associated with one or more of the non-Mendelian genes of interest, and outputting a phenotypic risk score for the subject.

In some aspects related to systems or non-transitory machine-readable media, the second dataset comprises genotype population data and phenotype population data for two or more blood relatives. In some aspects, the blood relative in the first dataset comprises one or more of the subject's mother, father, brother, sister, son, daughter, grandfather, grandmother, aunt, uncle, niece, nephew, and first cousin. In some aspects, the second dataset includes two or more subjects having the same blood relationship as the subjects in the first dataset. In some aspects, one or more of the blood relatives is a male relative. In some aspects, one or more of the blood relatives is a female relative.

In some aspects related to systems or non-transitory machine-readable media, the first dataset includes data for more than one blood relative of the subject. In some aspects, one or more of the blood relatives is a male relative and one or more of the blood relatives is a female relative.

In some aspects related to systems or non-transitory machine-readable media, the gene of interest is a genetic variant of interest.

In some aspects related to systems or non-transitory machine-readable media, the first dataset and second dataset include data associated with the age of onset of the phenotype.

Also provided are methods for outputting a polygenic risk score, the method comprising: receiving, from a first dataset, (i) genotype data for a subject having one or more non-Mendelian genes of interest and (ii) genotype data and phenotype data for one or more blood relatives of the subject that have one or more of the non-Mendelian genes of interest; receiving, from a second dataset, genotype population data and phenotype population data, wherein the population comprises one or more sets of two or more blood relatives; training a model on the first and second datasets to predict a risk in the subject based on the one or more non-Mendelian genes of interest, and outputting a polygenic risk score for the subject. Some aspects comprise training a model on the first and second datasets to predict how the risk in the subject is modified by one or more non-Mendelian genes of interest, relative to the risk in the subject given the phenotype data of the blood relatives.

Also provided are methods of treating a subject based on a phenotypic risk score.

### BRIEF DESCRIPTION OF DRAWINGS

FIG. 1 sets forth a simulated histogram of an expressed phenotype with a mean age of incidence of 60 years.

FIG. 2 is a block diagram of an example computing device.

FIG. 3 is the result of a simulation illustrating an aspect of the method applied to three genes where the third gene has population frequency of 1.0%; FIGS. 3A and 3B show histograms of predictions for subjects in which only a subset of relevant genes is available in the model; FIG. 3C shows a histogram of predictions for subjects in which all genetic variables are included.

FIG. 4 is the result of a simulation illustrating an aspect of the method applied to three genes where the third gene has population frequency of 0.2%; FIGS. 4A and 4B show histograms of predictions for subjects in which only a subset of relevant genes is available in the model; FIG. 4C shows a histogram of a predictions for subjects in which all genetic variables are included.

FIG. 5 is the result of a simulation illustrating an aspect of the method applied to three genes where the third gene has population frequency of 0.05%; FIGS. 5A and 5B show histograms of predictions for subjects in which only a subset of relevant genes is available in the model; FIG. 5C shows a histogram of predictions for subjects in which all genetic variables are included.

### DETAILED DESCRIPTION

Technical and scientific terms used herein have the meanings commonly understood by one of ordinary skill in the art to which the present invention pertains, unless otherwise defined. Materials to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “blood relatives” refers to two or more subjects who have one or more common ancestors. Non-limiting examples of a blood relative of a subject include the subject's mother, father, brother, sister, son, daughter, grandfather, grandmother, aunt, uncle, niece, nephew, and/or first cousin. In some aspects, the blood relative is a male. In some aspects, the blood relative is a female.

The term “gene” relates to stretches of DNA or RNA that encode a polypeptide or that play a functional role in an organism. A gene can be a wild-type gene, or a variant or mutation of the wild-type gene. A “gene of interest” refers to a gene, or a variant of a gene, that may or may not be known to be associated with a particular phenotype, or a risk of a particular phenotype.

“Expression” refers to the process by which a polynucleotide is transcribed from a DNA template (such as into a mRNA or other RNA transcript) and/or the process by which a transcribed mRNA is subsequently translated into peptides, polypeptides, or proteins. Where a nucleic acid sequence encodes a peptide, polypeptide, or protein, gene expression relates to the production of the nucleic acid (e.g., DNA or RNA, such as mRNA) and/or the peptide, polypeptide, or protein. Thus, “expression levels” can refer to an amount of a nucleic acid (e.g. mRNA) or protein in a sample.

Described are novel and unpredictable methods of using genetic information to determine the risk a subject will have a phenotype. For non-Mendelian genes, the probability of a subject developing a phenotype can be computed from population data. However, if a subject has a gene mutation that is the same mutation as one of their relatives, and that relative has the phenotype, the probability of the subject developing the phenotype can be computed more precisely than using the population risk computed without relatives' data.

### Gene Selection

The gene of interest can be identified by any means known in the art. For instance, the gene of interest can be selected based on a subject's personal genome. In some aspects, the gene of interest is a known non-Mendelian gene. In some aspects the gene of interest is a genetic variant of interest. In some aspects, the gene of interest has not independently been statistically significantly associated with an observed phenotype. In some aspects, the gene of interest is known to be associated with an observed phenotype.

### Dataset Selection

Datasets for determining risk can be obtained by any means known in the art. For instance, a first dataset can include genotype data and phenotype data for a subject and also for one or more blood relatives of the subject. The genotype data can include expression data for one or more genes of interest. The phenotype data can include observable characteristics or traits of a disease, including particular symptoms of the disease, or observable characteristics of a subject that are not associated with any disease.

The first dataset can be prepared by detecting the expression of one or more genes of interest in a subject and in one or more blood relatives of the subject. In some aspects, genotype data and/or phenotype data from a subject and from one or more blood relatives of the subject are acquired from a plurality of sources.

In some aspects, the first dataset further comprises information related to the age of the subject and/or the blood relatives. In some aspects, the first dataset comprises information related to the age of onset of a phenotype (e.g., a disease or condition, or particular symptoms associated with a disease or condition) in the subject and/or blood relatives of the subject.

In some aspects, the subject has a particular phenotype. In some aspects, the subject does not have the phenotype. In some aspects, the subject harbors one or more genes of interest. In some aspects, the subject does not harbor a gene of interest. In some aspects, one or more blood relatives of the subject harbor one or more of the genes of interest, and display a phenotype that is also observed in the subject. In some aspects, one or more of the blood relatives of the subject harbor one or more of the genes of interest, and display a phenotype that is not observed in the subject. In some aspects, one or more of the blood relatives of the subject harbor one or more of the genes of interest, and display a phenotype that is also observed in the subject. In some aspects, one or more of the blood relatives of the subject do not harbor one or more of the genes of interest, and display a phenotype that is not observed in the subject.

A second dataset can be used that has genotype population data and phenotype population data. Such population data for non-Mendelian genes can be used to determine the probability of a subject developing a phenotype. In some aspects, the population data includes data from two or more blood relatives. In some aspects, the population data includes data from one or more sets of two or more blood relatives, e.g., 2 sets, 3 sets, 4 sets, 5 sets, 10 sets, or more of blood relatives. The relation between the blood relatives can be the same as, different from, or overlapping with the relation between the subject and blood relative in the first dataset. In some aspects, the two or more blood relatives from the population data are not blood relatives to subjects used for the first dataset. In some aspects, the data for the second dataset is compiled from one or more publicly available databases. Non-limiting examples of such databases may include the United Kingdom (UK) Biobank; various genotype-phenotype datasets that are part of the Database of Genotype and Phenotype (dbGaP) maintained by the National Center for Biotechnology Information (NCBI); The European Genome-phenome Archive; OMIM; GWASdb; PheGenl; Genetic Association Database (GAD); and PhenomicDB.

The datasets can be compiled using data from one or more of a variety of tissues or body fluids. For instance, the first and/or second dataset can independently include data associated with brain tissue, heart tissue, lung tissue, kidney tissue, liver tissue, muscle tissue, bone tissue, stomach tissue, intestines tissue, esophagus tissue, and/or skin tissue, or any combination of such tissues. Additionally or alternatively, the datasets can include data associated with biological fluids, such as urine, blood, plasma, serum, saliva, semen, sputum, cerebral spinal fluid, mucus, sweat, vitreous liquid, and/or milk, or any combination of such fluids.

In some aspects the datasets are compiled using data from subjects having a particular condition or conditions, and/or a particular symptom or symptoms. In some aspects, the datasets are compiled using samples from a plurality of tissues and/or a plurality of biological fluids.

### Phenotypic Risk Score

Some aspects comprise determining a phenotypic risk score for the subject. A phenotypic risk score can indicate the likelihood that subject will develop a particular phenotype (e.g., a disease or condition, or a symptom of a disease or condition). The polygenic risk score can be determined using machine learning (including supervised and/or unsupervised machine learning algorithms). In some aspects, the polygenic risk score can be calculated by training a model on a first dataset (e.g., having genotype data and phenotype data for a subject and one or more blood relatives of the subject) and a second dataset (e.g., having genotype population data and phenotype population data). In some aspects, the training includes normalization (e.g., normalizing transcript expression levels of genes of interest to expression levels of housekeeping genes) and/or standardization steps (e.g., via SVM to scale transcript abundance to zero mean).

In some aspects, the phenotypic risk score is determined using resampling techniques, such as oversampling or undersampling. Some aspects comprise using binning and/or bagging techniques. In some aspects, parametric and/or non-parametric statistical tests are used to evaluate expression differences between subjects.

In some aspects, a phenotypic risk score can be used to classify a subject as being at risk of a phenotype. Classification can be performed using, for instance, SVM, logistic regression, random forest, nave bayes, and/or adaboost. In some aspects, the phenotypic risk score is a probability that the subject will develop a phenotype. In some aspects, the phenotypic risk score is a probability that the subject will develop a phenotype by a particular age.

In some aspects, the phenotypic risk score is determined using an area under the curve (AUC) measurement. For instance, the AUC can be more than about 0.5, more than about 0.55, more than about 0.6, more than about 0.65, more than about 0.7, more than about 0.75, more than about 0.8, more than about 0.85, more than about 0.9, more than about 0.95, more than about 0.97, more than about 0.98, or more than about 0.99.

### Implementation Systems

The methods described here can be implemented on a variety of systems. For instance, in some aspects the system for determining a phenotypic risk score includes one or more processors coupled to a memory. The methods can be implemented using code and data stored and executed on one or more electronic devices. Such electronic devices can store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals such as carrier waves, infrared signals, digital signals).

The memory can be loaded with computer instructions to train the model to determine a phenotypic risk score. In some aspects, the system is implemented on a computer, such as a personal computer, a portable computer, a workstation, a computer terminal, a network computer, a supercomputer, a massively parallel computing platform, a television, a mainframe, a server farm, a widely-distributed set of loosely networked computers, or any other data processing system or user device.

The methods may be performed by processing logic that comprises hardware (e.g. circuitry, dedicated logic, etc.), firmware, software (e.g., embodied on a non-transitory computer readable medium), or a combination of both. Operations described may be performed in any sequential order or in parallel.

Generally, a processor can receive instructions and data from a read only memory or a random access memory or both. A computer generally contains a processor that can perform actions in accordance with instructions and one or more memory devices for storing instructions and data. Generally, a computer will also include, or be operatively coupled to receive data from or transfer data to, or both, one or more mass storage devices for storing data, e.g., magnetic disks, magneto optical disks, optical disks, or solid state drives. However, a computer need not have such devices. Moreover, a computer can be embedded in another device, e.g., a smart phone, a mobile audio or media player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including, by way of example, semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

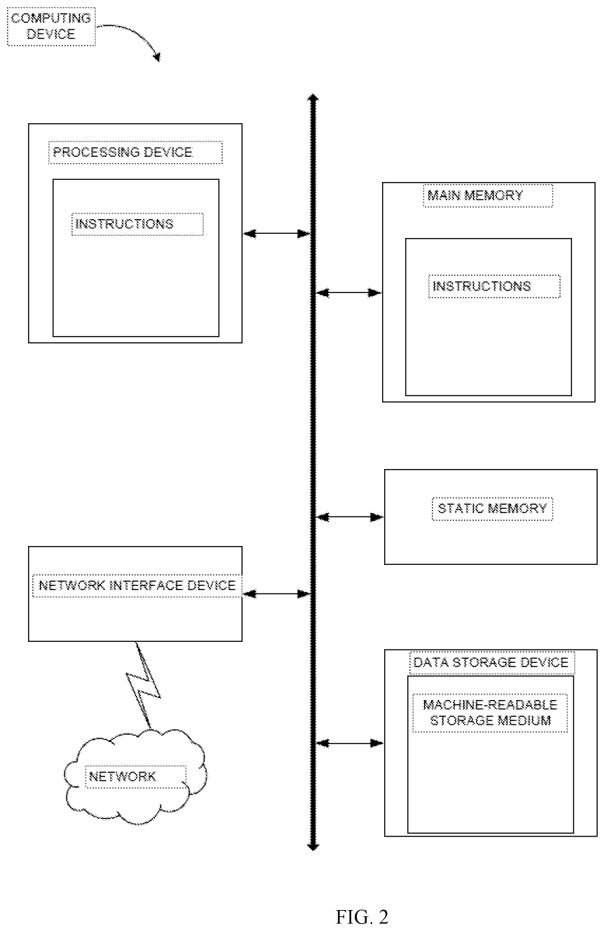

An exemplary implementation system is set forth in FIG. 2. Such a system can be used to perform one or more of the operations described here. The computing device may be connected to other computing devices in a LAN, an intranet, an extranet, and/or the Internet. The computing device may operate in the capacity of a server machine in client-server network environment or in the capacity of a client in a peer-to-peer network environment.

### Diagnosis and Treatment

In some aspects, a subject (e.g., a human subject) is diagnosed as having a condition or disease, or being at risk of having the condition or disease, based on the phenotypic risk score. For instance, in some aspects a subject having a particular phenotypic risk score is diagnosed as having the condition or disease. In some aspects, a subject having a particular phenotypic risk score is determined to be at increased risk of developing the condition or disease, or one or more symptoms thereof.

Some aspects comprise treating a subject determined to have, or be at increased risk of a condition or disease, or one or more symptoms of the disease or condition. The term “treat” is used herein to characterize a method or process that is aimed at (1) delaying or preventing the onset or progression of a disease or condition; (2) slowing down or stopping the progression, aggravation, or deterioration of the symptoms of the disease or condition; (3) ameliorating the symptoms of the disease or condition; or (4) curing the disease or condition. A treatment may be administered after initiation of the disease or condition. Alternatively, a treatment may be administered prior to the onset of the disease or condition, for a prophylactic or preventive action. In this case, the term “prevention” is used. In some aspects the treatment comprises administering a drug product listed in the most recent version of the FDA's Orange Book, which is herein incorporated by reference in its entirety. Exemplary conditions and treatments are also described PHYSICIANS' DESK REFERENCE (PRD Network 71st ed. 2016); and THE MERCK MANUAL OF DIAGNOSIS AND THERAPY (Merck 20th ed. 2018), each of which are herein incorporated by reference in their entirety.

The following examples are provided to illustrate the invention, but it should be understood that the invention is not limited to the specific conditions or details of these examples.

### EXAMPLES

### Example 1: Refining Risk Using Relatives' Information

As a simplified illustrative example, a possible mutation m on gene g was considered, with X_{gm} being a binary indicator variable where X_{gm}=1 if the mutation is present and X_{gm}=0 if the mutation is absent. For efficiency, X_{gm} was used interchangeably to refer to the mutation, the genetic locus of the mutation, and as the indicator of whether or not the mutation is present at that locus. In the subpopulation with the mutation X_{gm}, the phenotype arises with a probability of P(X_{gm})=p_{gm} (this notation will be used throughout the following examples). One way p_{gm} can be measured from studies is

$${p}_{gm} = \frac{{N}_{gm , affected}}{{N}_{gm , affected} + {N}_{gm , uaffected}}$$

where N_{gm,affected} and N_{gm,unaffected} are the number of subjects (e.g., people) with X_{gm} mutated who do and don't have the phenotype respectively.

It is assumed for this illustrative example that only one other mutation besides X_{gm} is known to affect the phenotype (e.g., mutation n and gene h, Xₕₙ) and Xₕₙ is at an unknown location in the genome assumed to not be in linkage disequilibrium with X_{gm}. For this example, it is assumed that Xₕₙ acts like a switch in that if X_{gm} and Xₕₙ are mutated then a subject will develop the phenotype but if only X_{gm} or Xₕₙ are mutated then the subject will not. If a mother and a child have X_{gm} mutated, and the mother has the phenotype, then the child's risk can be predicted more precisely than if the risk is determined based on subpopulation studies as p_{gm}. For this example, it is assumed that mutation Xₕₙ is rare enough that the probability of receiving this mutation from the father or the mother having more than one copy can be ignored. The chance that the child will develop the phenotype is thus roughly 50% because there is a 50% chance that the child inherits Xₕₙ mutation from the mother. Assume for this illustrative example that the general population risk is around 1% for the phenotype and mutation X_{gm} is a rare mutation that increases risk by 50%, increasing risk to roughly 1.5% for an individual who has mutation X_{gm} in which data from blood relatives is not included. If a child has X_{gm} mutated, and it is known that the mother has X_{gm} mutated and has the phenotype, the child's risk is now 50% instead of 1.5%. So, even for a moderate risk increase of 50%, given the simplified scenario of Xₕₙ acting as a switch for X_{gm}, the effect of the knowledge of the mother having the mutation and the phenotype is substantial.

In the scenario that one doesn't know all the mutations that interact with X_{gm} to affect the phenotype, or their mechanisms of interaction, the concept outlined above can be applied to empirically estimate the probability of a subject developing a phenotype if a blood relative has the same mutation and the associated phenotype. This involves extracting information from genotype-phenotype databases to calculate risk specific to a particular relative relationship and a particular mutation or gene. Assume a subject shares mutation X_{gm} with blood relative r where r may be mother, father, brother, sister, son, daughter, grandfather, grandmother, aunt, uncle, niece, nephew, first cousin female, first cousin male etc. Assume for now that the subject is at an age before the phenotype is likely to express, a lifetime risk of the subject can be considered without adjusting for the effects of the subject's current age (which can separately be incorporated, as discussed below). Find the number of people in the database N_{gm,r} that have the mutation X_{gm}, that have a relative r with the mutation X_{gm} and the phenotype, and that have that have either passed away or are at an age by which the phenotype will have developed if it will develop in that person (so that full lifetime risk can be calculated). Then find the number of people out of N_{gm,r} who were affected by the phenotype N_{gm,r,affected}. The estimated probability of the subject developing the phenotype is then:

$${\hat{p}}_{gm , r} = \frac{{N}_{gm , r , affected}}{{N}_{gm , r}}$$

### Example 2—Managing Limited Data

For a normal approximation of the binomial distribution one can use an exact binomial for small numbers the variance of the estimate of \hat{p}_{gm,r} is found:

$${{\hat{σ}}_{gm , r}}^{2} = \frac{{\hat{p}}_{gm , r} ( 1 - {\hat{p}}_{gm , r} )}{{N}_{gm , r}}$$

p_{gm} represents the probability of developing the phenotype given mutation X_{gm}, independent of information on relatives. \hat{p}_{gm,r} can be used if it is different from p_{gm} with sufficient confidence, e.g., two standard deviations, i.e. if

|p_{gm}−\hat{p}_{gm,r}|>2\hat{σ}_{gm,r}

Or, if an empirical estimate of p_{gm} has also been found:

$${\hat{p}}_{gm} = \frac{{N}_{gm , affected}}{{N}_{gm}} , \\ {{\hat{σ}}_{gm}}^{2} = \frac{{\hat{p}}_{gm} ( 1 - {\hat{p}}_{gm} )}{{N}_{gm}}$$

The following criterion can be used:

|\hat{p}_{gm}−\hat{p}_{gm,r}|>2\sqrt{\hat{σ}_{gm}²+\hat{σ}_{gm,r}²}

Or \hat{p}_{gm,r} can be adjusted some number of standard deviations in the direction of p_{gm} for the sake of conservatism: E.g. Using 2-sigma adjustment, if \hat{p}_{gm,r}>p_{gm}, then \hat{p}_{gm,r}→max(p_{gm}, \hat{p}_{gm,r}−2\hat{σ}_{gm,r}). Another approach is to break up the database into multiple sub-databases and upper-bounding the variance in the estimate of \hat{p}_{gm,r} empirically by calculating \hat{p}_{gm,r} for each sub-database and computing the sample variance.

One can also use test databases that are not used in the calculation of \hat{p}_{gm,r.} For example, one can identify all subjects in the test data who have mutation X_{gm}, and who have passed away. Then, \hat{p}_{gm,r} can be computed for each of these subjects using the training data, and compared to whether the subjects did or did not develop the phenotype to determine whether \hat{p}_{gm,r} which incorporates the relative information provides a more accurate prediction than p_{gm}.

### Example 3: Combining Similar Relative Relationships

Another approach is to combine the data on the male and female relatives, with the assumption that genes present on the X chromosome and not present on the Y chromosome have minimal effect on expression of the phenotype.

Furthermore, one can combine information from relatives that share a similar amount of genetic material with the subject of interest. In that case, let r designate each group of relatives that share the same amount of genetic information with the subject. The counts for each group r will be pooled. Namely, using a similar approach as described above, N_{gm,r} would now represent the number of people in the database that have the mutation X_{gm} and that have a relative in the group r, with the mutation X_{gm} and the phenotype; N_{gm,r,affected} would now represent the number out those who are affected. For example, r=½ represents the group with half the subject's genetic information—mother, father, brother, sister, son, daughter; r=¼ for the group with one quarter the genetic information grandfather, grandmother, half-brother, half-sister, aunt, uncle, niece, nephew, grandson, granddaughter etc.; r=⅛ for the group with one eighth the genetic information etc. In this approach, any two subjects who have relatives that have X_{gm} and the phenotype, and are in the same relative group r, would have the same \hat{p}_{gm,r}. This same approach can be applied to group relatives according to whether they share the same amount of genetic information as the subject and are of the same gender as other members of the group. In this case, for example, the group with ¼ the genetic information as the subject would be broken into a male group: grandfather, half-brother, uncle, nephew, grandson etc. and a female group: grandmother, half-sister, aunt, niece, granddaughter etc. Many different combinations or sets of relatives may be used, as designated by r, and many different subsets of the relatives in that set who have X_{g} may be required to have the phenotype, rather than simply one or more, to include the subject in the count N_{gm,r}.

### Example 4: Gene Level Mutations

Another approach is to address the presence of a mutation at the gene level rather than treat each variant in isolation. Namely, let X_{g} represent a mutated gene g, which incorporates all the mutations X_{gm}, m=1 M which are known to have the same effect on the function gene g such as, for example, a loss of function. In this case, one can count N_{g,r}, which is the number of people who have a loss of function mutation in gene g and a relative in group r that also have a mutation of that type, such as a loss of function mutation, in gene g. The probabilities at the gene level can then be calculated:

$${\hat{p}}_{g , r} = \frac{{N}_{g , r , affected}}{{N}_{g , r}} , \\ {{\hat{σ}}_{g , r}}^{2} = \frac{{\hat{p}}_{g , r} ( 1 - {\hat{p}}_{g , r} )}{{N}_{g , r}}$$

### Example 5: Incorporating Age

Another approach addresses the age of people in the database and eliminates the need to only consider people who have died in computing N_{gm,r}. Working at the level of a gene rather than a mutation, one can calculate N_{g,r} instead of N_{gm,r}.

Let \hat{p}_{g,r}(A) be the estimate of probability that subject of age A, mutation X_{g} and relative r with mutation X_{g}, develops the phenotype if they do not currently have the phenotype. Depending on the availability of data, one may or may not incorporate the requirement that the relatives with mutation X_{g} have expressed or will express the phenotype. Let N_{g,r,A} be all subjects with mutation X_{g}, and relative r with mutation X_{g}, who lived longer than age A and did not have the phenotype at age A. Let N_{g,r,A,affected} be the number of those N_{g,r,A} subjects who expressed the phenotype from age A onwards.

$${\hat{p}}_{g , r} ( A ) = \frac{{N}_{g , r , A , affected}}{{N}_{g , r , A}} , \\ {{\hat{σ}}_{g , r} ( A )}^{2} = \frac{{\hat{p}}_{g , r} ( A ) ( 1 - {\hat{p}}_{g , r} ( A ) )}{{N}_{g , r}}$$

Note that there are many other ways to approximate p_{g,r}(A) for a subject that has not yet developed the phenotype, without changing the essential concept. For example, for limited data, one can approximate p_{g,r}(A) by computing pᵣ(A) or p_{g}(A), i.e. not filtering subjects in the database based on requiring them to have mutation X_{g} or have relative r with the mutation X_{g}.

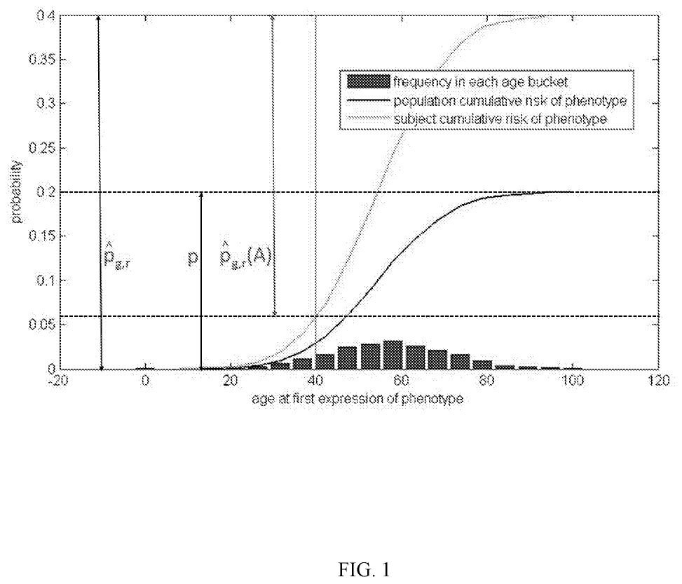

Another approach, with limited data, is to consider all people in the database who expressed the phenotype, independent of whether they have mutation X_{g} or relative r, and compute the histogram of when the phenotype was expressed. Such a simulated example histogram is shown in bars in the FIG. 1 for a phenotype with mean age of incidence 60 years. The cumulative probability of an individual expressing the phenotype as a function of age can be computed, shown in red, which asymptotes to p, the population frequency of expressing the phenotype, in this case p=0.2. One can make the approximation that for individual subjects with risks that are different to p, the relative probabilities for the age at which the phenotype is likely to express is unchanged. In that case, for a subject with estimated lifetime risk \hat{p}_{g,r}, one may simply scale the cumulative probability by

$$\frac{{\hat{p}}_{g , r}}{p} .$$

In the example, the cumulative probability for the subject is shown with the gray line which asymptotes at \hat{p}_{g,r}=0.4. Using an approximating assumption, this is still a cumulative probability distribution for an underlying probability distribution with mean 60 years. For a subject at age A, \hat{p}_{g,r}(A) can be found by determining how much more probability the subject has yet to accumulate in their lifetime, shown as the vertical line at age A=40, \hat{p}_{g,r}(40)=0.34 in the example in the figure. Many variations on this theme are possible without changing the essential concept, using other assumptions and probability distributions derived from population genetics and epidemiology, adjusted by age for the subjects.

### Example 6: Combing the Effect of Multiple Relatives

Another approach involves a situation where a subject has multiple relatives that have the variant and the phenotype. The simplest approach is to use the same method as above, but rather than count cases in a database that have only the one relative, count all cases that have the same set of multiple relatives, where a relative is classified in terms of the groupings r described above, such has sharing the same amount of genetic data in common with the subject and being a particular gender. For example, if one groups by gender as well as by amount of genetic information in common, a subject that has one father, one uncle, and one grandfather who all have the variant and the disease can be counted along with a subject that has, say, two sons and one uncle that have the variant and the disease. As another example, if one only groups by amount of genetic information in common, a subject that has one father, one aunt, and one grandmother who all have the variant and the disease can be counted along with a subject that has, say, two sons and one uncle that have the variant and the disease.

In the case of limited data, the risk can be approximated, which will typically result in a lower bound, by ignoring some of the subject's relatives who have the variant and disease, so that more data can be pooled. In this case, one would typically prioritize those relatives that share more genetic information with the subject. For example, a subject that has one father, one uncle, and one grandfather who all have the variant and the disease can be treated as a subject that has only one relative, a father, that has the variant and the disease.

Another approach combines the data across several categories of relatives. There are many empirical or heuristic approaches to this concept. For instance, one exemplary approach is relevant if the number of genes effecting the penetrance of X_{g} is very large, and the individual effect size of each of these genes is very small. Let Δ\hat{p}_{g,r} represent the difference from the established probability p_{g} if one inherits all of the relevant mutated genes from a relative. Now, one can make the highly simplifying and non-accurate assumption that the change in probability would scale proportionately to the number of relevant mutated genes inherited

\hat{p}_{g,r}−p_{g}=rΔ\hat{p}_{g,r}, where r=½,¼,⅛ . . . as described above for each relative group.

Then one may solve for Δ\hat{p}_{g,r} using a set of equations for each relative group, which can be weighted by each group's respective variance:

$$Δ {\hat{p}}_{g , r} = \frac{{\sum }_{r = \frac{1}{2} , \frac{1}{4} , \frac{1}{8} … } \frac{r}{{{\hat{σ}}_{g , r}}^{2}} ( {\hat{p}}_{g , r} - {p}_{g} )}{{\sum }_{r = \frac{1}{2} , \frac{1}{4} , \frac{1}{8} … } \frac{{r}^{2}}{{{\hat{σ}}_{g , r}}^{2}}}$$

One may then use Δ\hat{p}_{g,r} and known p_{g} to estimate \hat{p}_{g,r}.

### Example 7: Applying the Method to Polygenic Risk Scores

The techniques described above can be used in the context of polygenic risk scores, or regression models describing the probability of developing phenotypes, or in other machine learning models for determining the probability of a phenotype. For example, one can model a phenotype based on the polygenic, or multivariate, regression models below, at the mutation or the gene level:

P=b₀+Σ_{g=1 . . . G}Σ_{m=1 . . . M}_{_{g}}b_{gm}X_{gm}

P=b₀+Σ_{g=1 . . . G}b_{g}X_{g}

Assume indicator variable X_{g} at the gene level, as described previously, combines all mutations X_{gm} of similar type, such as loss of function, or particular types of gain of function. X_{g}=1 if the gene has a mutation and X_{g}=0 if not. This same concept can be extended to different classifications of mutations such as loss of function or different classes of gain of function mutations.

The below example works at the mutation level, with no loss of generality.

Regression models such as the above can be adjusted based on the probabilities derived for a particular individual using the methods outlined herein. Consider the case where P is a Polygenic Risk Score (PRS) that is not a probability per se, but has meaning in relation to other scores, such as for determining in what percentile a subject's genetic risk score lies. In this case, one can set the bias parameter b₀=0 and the others to the effect size of each gene or variant. This effect size b_{gm} can be estimated by taking the log of the ratio of the probabilities of developing the disease phenotype, D, with and without the mutation X_{gm}.

$${b}_{gm} = log ( \frac{P ( D \vert {X}_{gm} )}{P ( D \vert \overline{{X}_{gm}} )} )$$

P(D|X_{gm}) is the probability of the disease given the mutation and is approximated by the probability calculated above P(D|X_{gm})=\hat{p}_{gm}. To calculate P(D|X̅_̅{̅g̅m̅}̅) use the expansion:

P(D)=P(D|X_{gm})P(X_{gm})+P(D|X̅_̅{̅g̅m̅}̅)P(X̅_̅{̅g̅m̅}̅)

Replacing P(X̅_̅{̅g̅m̅}̅)=1 P(X_{gm}) and substituting into P(D|X̅_̅{̅g̅m̅}̅) into the above, one gets:

$${b}_{gm} = log ( \frac{P ( D \vert {X}_{gm} ) ( 1 - P ( {X}_{gm} ) )}{P ( D ) - P ( D \vert {X}_{gm} ) P ( {X}_{gm} )} )$$

$${b}_{gm} = log ( \frac{{\hat{p}}_{gm} ( 1 - P ( {X}_{gm} ) )}{P ( D ) - {\hat{p}}_{gm} P ( {X}_{gm} )} )$$

where P(X_{gm}) is the frequency of the mutation in the population, P(D) is the frequency of the phenotype in the population, previously defined as p. P(D) is used here for clarity. One approach is to set the model parameters to the log of the odds ratio. When the mutation is rare in the population, i.e. P(X_{gm}) is small, this simplifies to

$${b}_{gm} \approx log ( \frac{{\hat{p}}_{gm}}{P ( D )} ) = log ( \frac{{\hat{p}}_{gm}}{p} )$$

which is what is often used in practice. When \hat{p}_{gm} is close to p, in that the particular variant X_{gm} effect size is small, as is typically the case, one can use

$${b}_{gm} \approx \frac{{\hat{p}}_{gm}}{p} - 1$$

If it is known that the individual of interest has affected relative(s) r, the parameters can be changed to take this into account using an effect size relative to pᵣ, the probability that one will develop the phenotype given affected relative(s) r.

$${b}_{gm , r} \approx log ( \frac{{\hat{p}}_{gm , r}}{{p}_{r}} ) - 1$$

where \hat{p}_{gm,r} is as described above. We will describe below why these parameters are defined relative to pᵣ rather than p, and what the advantages of this approach are. But first note that there are many variations of this concept. For example, we can weight the parameters by the inverse of their variance:

$$var ( {b}_{gm , r} ) \approx \frac{1}{{{p}_{r}}^{2}} var ( {\hat{p}}_{gm , r} ) = \frac{{{\hat{σ}}_{gm , r}}^{2}}{{{p}_{r}}^{2}}$$

$$So$$

$${b}_{gm , r , weighted} = \frac{{{p}_{r}}^{2}}{{{\hat{σ}}_{gm , r}}^{2}} ( \frac{{\hat{p}}_{gm , r}}{{p}_{r}} - 1 )$$

In order to understand why the parameters are defined relative to pᵣ rather than p, consider that a polygenic model is attempting to model the probability of a phenotype resulting from multiple genetic variables. Assume for now that there are three genetic variables X₁, X₂, X₃ as follows

$$P ( D \vert {X}_{1} {X}_{2} {X}_{3} ) = \frac{P ( {DX}_{1} {X}_{2} {X}_{3} )}{P ( {X}_{1} {X}_{2} {X}_{3} )} = \frac{P ( {X}_{1} \vert {DX}_{2} {X}_{3} ) P ( {DX}_{2} {X}_{3} )}{P ( {X}_{1} {X}_{2} {X}_{3} )} = \frac{P ( {X}_{1} \vert {DX}_{2} {X}_{3} ) P ( {DX}_{2} {X}_{3} )}{P ( {X}_{1} {X}_{2} {X}_{3} )}$$

But if one makes assumption that X₁, X₂ and X₃ are approximately independent then P(X₁|DX₂X₃)≈P(X₁|D), and P(X₁X₂X₃)≈P(X₁)P(X₂)P(X₃) hence

$$P ( D \vert {X}_{1} {X}_{2} {X}_{3} ) \approx \frac{P ( {X}_{1} \vert D ) P ( {DX}_{2} {X}_{3} )}{P ( {X}_{1} ) P ( {X}_{2} ) P ( {X}_{3} )}$$

where P(DX₂X₃) can be decomposed due to independence assumptions

$$P ( {DX}_{2} {X}_{3} ) \approx P ( {X}_{2} \vert {DX}_{3} ) P ( {DX}_{3} ) \approx \frac{P ( {X}_{2} \vert D ) P ( {DX}_{3} )}{P ( {X}_{1} ) P ( {X}_{2} ) P ( {X}_{3} )} = \frac{P ( {X}_{2} \vert D ) P ( {X}_{3} \vert D ) P ( D )}{P ( {X}_{1} ) P ( {X}_{2} ) P ( {X}_{3} )}$$

Substituting in the terms

$$P ( D \vert {X}_{1} {X}_{2} {X}_{3} ) = \frac{P ( {X}_{1} \vert D ) P ( {X}_{2} \vert D ) P ( {X}_{3} \vert D ) P ( D )}{P ( {X}_{1} ) P ( {X}_{2} ) P ( {X}_{3} )}$$

Now applying Bayes Rule where P(X₁|D)/P(X₁)=P(D|X₁)/P(D):

$$P ( D \vert {X}_{1} {X}_{2} {X}_{3} ) \approx P ( D ) \frac{P ( D \vert {X}_{1} ) P ( D \vert {X}_{2} ) P ( D \vert {X}_{3} )}{P ( D ) P ( D ) P ( D )}$$

This argument can apply to any number of variables X₁ . . . X_{G}. Is should also be noted that these independent variables need not be only genetic but could also be lifestyle or other phenotypes.

$$P ( D \vert {X}_{1} … {X}_{G} ) \approx P ( D ) \frac{P ( D \vert {X}_{1} ) P ( D \vert {X}_{2} ) … P ( D \vert {X}_{G} )}{P ( D ) P ( D ) … P ( D )}$$

$$logP ( D \vert {X}_{1} … {X}_{G} ) \approx logP ( D ) + log \frac{P ( D \vert {X}_{1} )}{P ( D )} + … log \frac{P ( D \vert {X}_{G} )}{P ( D )}$$

The description above for computing log P(D|X₁ . . . X_{G}) outlines the derivation and concept behind polygenic prediction models summing log odds ratios for each SNP, or approximations to the same, in order to estimate log P(D|X₁ . . . X_{G}). Each of the factors of the form

$$\frac{P ( D \vert {X}_{g} )}{P ( {X}_{g} )}$$

provides a theoretical background for use of odds ratio applied to genetic locus g in polygenic risk models. If X_{g}=1 then the baseline population probability P(D) is scaled by

$$\frac{P ( D \vert {X}_{g} = 1 )}{P ( D )}$$

but if X_{g}=0 then P(D) is scaled by

$$\frac{P ( D \vert {X}_{g} = 0 )}{P ( D )} .$$

This is similar to what is done in many PRS models, as mentioned above, where one computes an effect size b_{g}:

$${b}_{g} = log ( \frac{P ( D \vert {X}_{g} = 1 )}{P ( D \vert {X}_{g} = 0 )} )$$

and then computes a PRS score by summing the effect sizes according to the genetic data of the individual:

PRS=Σ_{g=1 . . . G}b_{g}X_{g}

When X_{g}=1, rather than scaling by

$$\frac{P ( D \vert {X}_{g} = 1 )}{P ( D )}$$

as described above, one is both adding log P(D|X_{g}=1) and subtracting log P(D|X_{g}=0). The difference between these two scenarios is not typically significant in practice, as one doesn't typically use PRS to directly infer probability of the disease. Rather, subjects will typically be bucketed into bins based on their PRS and each bin will be separately characterized with a particular risk based on counting the fraction of individuals in that bin who do in fact have the disease. Put differently, a mapping usually a linear mapping is typically created between PRS and the actual risk of an individual having the disease. Consequently, any scaling issues, or increasing of effect sizes, applied to computing PRS are not significant.

The purpose of the PRS or the estimation of P(D|X₁ . . . X_{g}) is to replicate as closely as possible the probability of disease or phenotype for the subject, and to differentiate as thoroughly as possible between subjects that have different probabilities of disease. To show the value of the use of relative information, one can use the more theoretical probability formulation in the explanation below and the MATLAB simulation code discussed below. Namely, the below explanation compares the efficacy of estimating P(D|X₁ . . . X_{g}) without using relative information, as is typically done, to the efficacy of estimating the probability of disease incorporating the relative information captured in variable Xᵣ.

In the derivation for estimating P(D|X₁ . . . X_{g}) above, several approximations were made based on strong assumptions about the independence of the variables X₁ . . . X_{g}. Now, let Xᵣ variable represent whether a relative or set of relatives have the disease or phenotype of interest. This variable is typically not independent of X₁ . . . X_{G}. For example, if these are genetic variables, the presence of an effected relative considerably impacts the probability of the subject having genes, or the probability that X₁=1, . . . , X_{G}=1. However, if instead of calculating the risk relative to the population average, P(D), one instead calculates the risk relative to the probability of having the disease or phenotype of interest, given a set of relatives who have the disease or phenotype P(D|Xᵣ), one can leverage the information contained in the family history to create a more powerful polygenic prediction model, without extending the assumption of independence in that context beyond the variables, X₁ . . . X_{G}. One can use the same derivation arguments as above for P(D|X₁X₂X₃), to calculate the risk given Xᵣ, using similar independence assumptions between X₁, X₂ and X₃ and without having to ignore the dependence between Xᵣ and X₁X₂ . . . X₃.

$$P ( ( D \vert {X}_{r} ) \vert {X}_{1} {X}_{2} {X}_{3} ) = P ( D \vert {X}_{r} {X}_{1} {X}_{2} {X}_{3} ) \approx P ( D \vert {X}_{r} ) \frac{P ( D \vert {X}_{r} {X}_{1} )}{P ( D \vert {X}_{r} )} \frac{P ( D \vert {X}_{r} {X}_{2} )}{P ( D \vert {X}_{r} )} \frac{P ( D \vert {X}_{r} {X}_{3} )}{P ( D \vert {X}_{r} )}$$

Similarly, one can extend this methodology to any number of genetic, lifestyle, environmental or phenotype variables X₁ . . . X_{G}. In the case for which one can assume independence between these variables:

$$P ( ( D \vert {X}_{r} ) \vert {X}_{1} {X}_{2} … {X}_{G} ) = P ( D \vert {X}_{r} {X}_{1} {X}_{2} … {X}_{G} ) \approx P ( D \vert {X}_{r} ) \frac{P ( D \vert {X}_{r} {X}_{1} )}{P ( D \vert {X}_{r} )} \frac{P ( D \vert {X}_{r} {X}_{2} )}{P ( D \vert {X}_{r} )} … \frac{P ( D \vert {X}_{r} {X}_{G} )}{P ( D \vert {X}_{r} )}$$

Similarly to what was described above, one approach is create a PRS is to compute the effect sizes b_{g,r} as follows:

$${b}_{g , r} = log ( \frac{P ( D \vert {X}_{r} {X}_{g} = 1 )}{P ( D \vert {X}_{r} {X}_{g} = 0 )} )$$

where P(D|XᵣX_{g}=1) and P(D|XᵣX_{g}=0) are computed from the empirical data. Then compute a PRS score for people who have the relevant affected relative or set of affected relatives, by summing:

PRS_{X}_{ᵣ}=Σ_{g=1 . . . G}b_{g,r}X_{g}

The explanation that follows will focus on the case of three genetic variables, which are approximated to be independent. A MATLAB simulation is described to illustrate the value of using the available data from the relatives Xᵣ to model P(D|XᵣX₁X₂X₃) rather than P(D|X₁X₂X₃), which will be less precise in its ability to model the probability of disease for each individual and will typically result in more false results, increased healthcare costs, poorer outcomes etc. The explanation that follows could equally make use of the formulation above for computing PRS_{X}_{ᵣ} instead of PRS, but it uses the more theoretically based estimation of P(D|X₁X₂X₃Xᵣ).

Consider an example where we have two genes X₁ and X₂, with respective incidence rates in the population of 1/20 and 1/50, and X₂ acts as a switch for X₁ so that a subject will have the phenotype if both X₁=1 and X₂=1. To make the example more illustrative, assume further that these are not the only factors that can cause the disease, but that there is another gene X₃ which causes the disease with 100% penetrance when present. Furthermore, we will assume without loss of generality of the concept that the set of relatives considered for each subject is just their parents, namely Xᵣ=1 if either parent has the disease and Xᵣ=0 if neither parent has the disease. The MATLAB code in Appendix A implements the invented concepts applied to this scenario. Note that the simulation uses the same data to create the model and test the model. This is because so few parameters are being estimated compared to the number of simulated subjects, and so one would obtain roughly the same results generating new test data. Namely, the reduction to practice in this MATLAB focuses on the versatility of each of the modeling approaches, or the ability of the models to accurately estimate the disease probability described above and captured in the data, rather than focus on the effects of limited data.

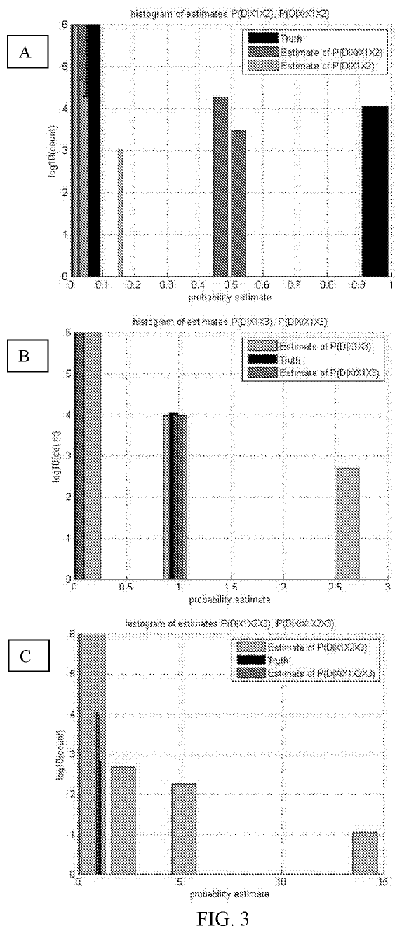

FIGS. 3A and 3B shows the histogram of predictions on a y axis log scale for each of the subjects when gene X₃ has frequency of 1/100 in the general population, and only a subset of the relevant genes are available in the model. Namely, FIG. 3A describes a model using only genetic variables X₁ and X₂ and FIG. 3B describes a model using only genetic variables X₁ and X₃. Such scenarios are often the case, for example, when a polygenic model only covers certain relevant SNPs in a subset of genes, whereas other relevant genes will not be included in the model. This arises, for example, because the excluded genetic variables don't reach statistical significance in a model that assumes linearity of effect and independence of the genetic variables, or because the excluded gene is affected by many rare variants that together have a significant effect but aren't associated with any one common variant with high enough frequency to be recognized as a SNP or “Single Nucleotide Polymorphism.” In both figures is included the truth for each of the subjects, namely whether each subject actually developed the disease or not, captured as 1 or 0 respectively. FIG. 3A illustrates the modeling of that data by estimating P(D|X₁X₂) and P(D|XᵣX₁X₂). FIG. 3B illustrates the modeling of that data by estimating P(D|X₁X₃) and P(D|XᵣX₁X₃). One can see, as is often the case, that the inclusion of the relative information enables the model to more closely capture the true underlying statistical model and more accurately emulate the truth. FIG. 3C illustrates the accuracy when all genetic variables are included, namely X₁ X₂ and X₃, resulting in estimates P(D|X₁X₂X₃) and P(D|XᵣX₁X₂X₃). FIG. 3C also assumes P(X₃)= 1/100.

Table 1 describes the Root-Mean-Square Error (RMSE) of several models from the simulation, using different combinations of genetic variables when different combinations of genes are used in a polygenic risk model, with and without information about the relatives Xᵣ which is the parents in this example.

TABLE 1
RMSE Estimate

| | Root Mean Square Error of Estimate | | |
|---|---|---|---|
| | P(X3) = 1/100 | P(X3) = 1/500 | P(X3) = 1/2000 |
| P(D\|XrX1) | 0.0769 | 0.0429 | 0.0330 |
| P(D\|X1) | 0.1041 | 0.0536 | 0.0383 |
| P(D\|XrX1X2) | 0.0769 | 0.0427 | 0.0317 |
| P(D\|X1X2) | 0.1030 | 0.0486 | 0.0251 |
| P(D\|XrX1X3) | 0.0313 | 0.0294 | 0.0288 |
| P(D\|X1X3) | 0.0509 | 0.0686 | 0.0800 |
| P(D\|XrX1X2X3) | 0.0312 | 0.0290 | 0.0279 |
| P(D\|X1X2X3) | 0.0846 | 0.0853 | 0.0540 |

In the latter case represented by FIG. 3C, the incorporation of the parent's disease history, namely Xᵣ, changes the RMSE from 0.0846 to 0.0312, or a 63% reduction.

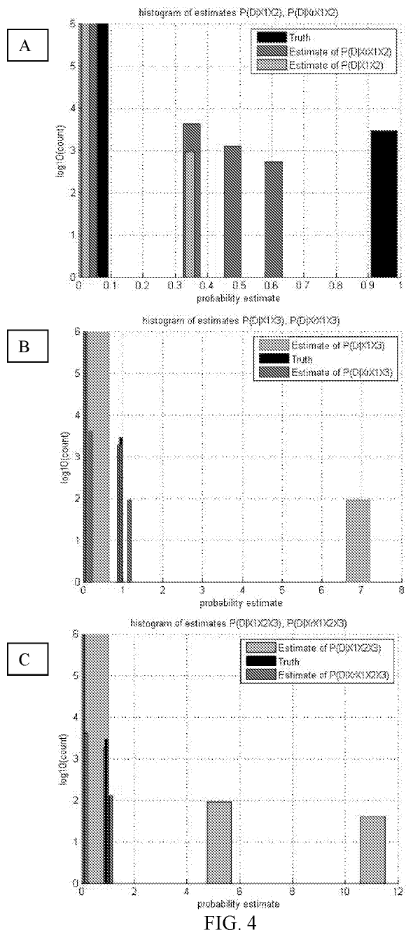

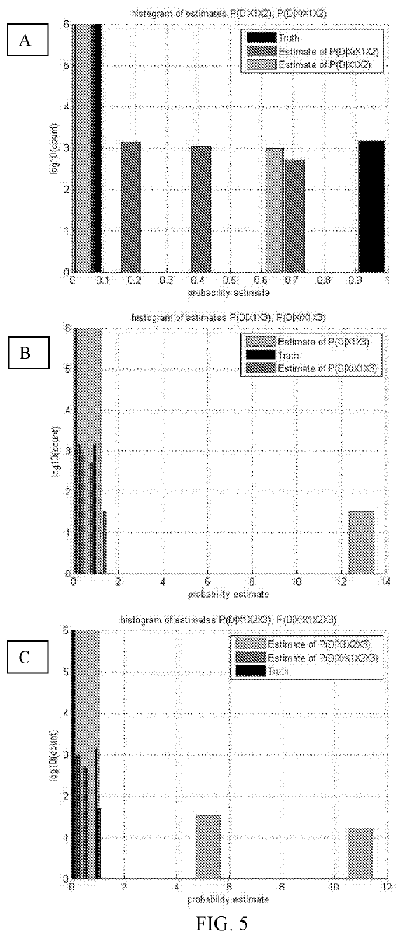

FIGS. 4A-C represents a similar situation to FIGS. 3A-3C, except that P(X₃)= 1/500. FIG. 5A-C represents a similar situation to FIGS. 3A-3C, except that P(X₃)= 1/2000. The RMSE for all of these scenarios described in the FIGS. 3, 4, and 5 are captured in Table 1, along with other scenarios. Note that in general the incorporation of the relative information Xᵣ generally improves performance in matching the truth data.

### Example 8: Other Approaches to Modeling Phenotype Probability

One can also modify the parameters for an individual using the approaches described herein when modeling the probability of a phenotype (rather than a risk score per se), for example using an approach based on logistic regression. At the gene level, a logistic regression model may be:

$$P ( D \vert {X}_{r} {X}_{1} … {X}_{G} ) = \frac{1}{1 + exp ( - {b}_{0} - {a}_{0} {\sum }_{g = 1 … G} {b}_{g , r} {X}_{g} )}$$

Where parameters a₀ and b₀ can be fitted to the data, having used concepts outlined above to select b_{g}.

The same concept can be applied to estimating P(D|XᵣX₁ . . . X_{G}) using nonlinear combinations of genes or variants. Here, again without loss of generality, we will work at the gene rather than the variant level. Assuming one wants to capture the interactions between genes and assuming that one is only looking at two gene interactions (the same concept can be applied, albeit with possible data challenges, to more than two gene interactions). One can create an independent variable for a regression model from any logical combination of the two genes X₁ and X₂: X₁X₂ (X₁ AND X₂), X̅₁̅X̅₂̅, X₁X̅₂̅, and X̅₁̅X₂. It should be born in mind, for regression models, that the presence of X₁ and X₂ in the set of independent variables will only require the use of two additional logical combinations as independent variables such as X₁X₂ and X̅₁̅X̅₂̅, since independent variables of other combinations such as X₁X̅₂̅ or X̅₁̅X₂ are linearly dependent on the variables already included. A model looking at gene interactions can be created with limited data, for example, by first building a linear regression model using standard methods, and then collecting all genes g=1 . . . G that are found to be significant and describing the nonlinear interaction of these genes. One may also use other machine learning methods, such as for example principal components, support vector machines, neural networks, deep-learning neural networks, and other functions to combine the genetic variables, to model P(D|XᵣX₁ . . . X_{G}).

### APPENDIX A: MATLAB FORMULA

% rel_sim
% simulates training polygenic prediction using relative relationships
% simulation parameters
n=1000000; % 1000000; % number of families
p_x1= 1/20; % 1/20; % P(X1) the probability of X1 variant in the general population
p_x2= 1/50; % 1/50; % P(X2) the probability of X2 variant in the general population
p_x3= 1/2000; % 1/100; % 1/500; % 1/2000; % P(X3) the probability of X3 variant in the general population
% setting up variables
% assume no denovo variants
% assume no homozygotes of variant in parents
% ph_x1=min(roots([1−2p_x1])); % probability per homolog; comment out if assume no homozygotes of variant in parents
% ph_x2=min(roots([1−2p_x2])); % probability per homolog; comment out if assume no homozygotes of variant in parents
% create parents
par1_vec_x1=(rand(n,1)<p_x1); % 1 if have variant 0 if don't
par1_vec_x2=(rand(n,1)<p_x2); % 1 if have variant 0 if don't
par1_vec_x3=(rand(n,1)<p_x3); % 1 if have variant 0 if don't
par2_vec_x1=(rand(n,1)<p_x1); % 1 if have variant 0 if don't
par2_vec_x2=(rand(n,1)<p_x2); % 1 if have variant 0 if don't
par2_vec_x3=(rand(n,1)<p_x3); % 1 if have variant 0 if don't
par1_vec_dis=(par1_vec_x1 & par1_vec_x2)|par1_vec_x3;
par2_vec_dis=(par2_vec_x1 & par2_vec_x2)|par2_vec_x3;
par_vec_dis=par1_vec_dis|par2_vec_dis;
% create children
p_inh_x1=0.5*par1_vec_x1+0.5*par2_vec_x1−0.25*par1_vec_x1.*par2_vec_x1;
chi_vec_x1=(rand(n,1)p_inh_x1);
p_inh_x2=0.5*par1_vec_x2+0.5*par2_vec_x2−0.25*par1_vec_x2.*par2_vec_x2;
chi_vec_x2=(rand(n,1)p_inh_x2);
p_inh_x3=0.5*par1_vec_x3+0.5*par2_vec_x3−0.25*par1_vec_x3.*par2_vec_x3;
chi_vec_x3=(rand(n,1)p_inh_x3);
chi_vec_dis=(chi_vec_x1 & chi_vec_x2) chi_vec_x3; % child gets sick if either (x1 and x2) or x3%
%%% train model for phenotype using standard method: P(D/X1X2)=P(D)*P(D/X1)/P(D)*P(D/X2)/P(D)*P(D/X3)/P(D)
% just using child data for now; can do this also for parents
p_dis_h=length(find(chi_vec_dis−1))/n
chi_vec_x1e1_ind=find(chi_vec_x1−1);
p_dis_x1e1_h=length(find(chi_vec_dis(chi_vec_x1e1_ind)−1))/length(chi_vec_x1e1_ind);
chi_vec_x1e0_ind=find(chi_vec_x1−0);
p_dis_x1e0 h=length(find(chi_vec_dis(chi_vec_x1e0_ind)−1))/length(chi_vec_x1e0_ind);
chi_vec_x2e1 ind=find(chi_vec_x2−1);
p_dis_x2e1 h=length(find(chi_vec_dis(chi_vec_x2e1 ind)−1))/length(chi_vec_x2e1 ind);
chi_vec_x2e0 ind=find(chi_vec_x2−0);
p_dis_x2e0 h=length(find(chi_vec_dis(chi_vec_x2e0 ind)−1))/length(chi_vec_x2e0 ind);
chi_vec_x3e1 ind=find(chi_vec_x3−1);
p_dis_x3e1 h=length(find(chi_vec_dis(chi_vec_x3e1 ind)−1))/length(chi_vec_x3e1 ind);
chi_vec_x3e0 ind=find(chi_vec_x3-0);
p_dis_x3e0 h=length(find(chi_vec_dis(chi_vec_x3e0 ind)−1))/length(chi_vec_x3e0 ind);
% prediction on the training data
% can also implement this on test data
p_dis_x1_h=zeros(n,1);
p_dis_x1_h(chi_vec_x1e1_ind)=p_dis_x1e1 h;
p_dis_x1_h(chi_vec_x1e0_ind)=p_dis_x1e0_h;
p_dis_x2_h=zeros(n,1);
p_dis_x2_h(chi_vec_x2e1 ind)=p_dis_x2e1 h;
p_dis_x2_h(chi_vec_x2e0 ind)=p_dis_x2e0 h;
p_dis_x3_h=zeros(n,1);
p_dis_x3_h(chi_vec_x3e1 ind)=p_dis_x3e1 h;
p_dis_x3_h(chi_vec_x3e0 ind)=p_dis_x3e0 h;
% prediction using x1 and x2
p_dis_x1x2_h=p_dis_h*(p_dis_x1_h/p_dis_h).*(p_dis_x2_h/p_dis_h);
% prediction using x1 and x3
p_dis_x1x3_h=p_dis_h*(p_dis_x1_h/p_dis_h).*(p_dis_x3_h/p_dis_h);
% prediction using x1,x2 and x3
p_dis_x1x2x3_h=p_dis_h*(p_dis_x1_h/p_dis_h).*(p_dis_x2_h/p_dis_h).*(p_dis_x3_h/p_dis_h);
%%%% train model for phenotype using relative method: P(D/Xr/X1X2)=P(D/Xr)*P(D/XrX1)/P(D/Xr)*P(D/XrX2)/P(D/Xr)
% just using child data for now to train; can train and test also for parents
par_vec_dis_ind=find(par_vec_dis−1);
p_dis_xr_h=length(find(chi_vec_dis(par_vec_dis_ind)−1))/length(par_vec_dis_ind);
% computing P(D/XrX1) for all states
chi_vec_xre1_x1e1_ind=find(par_vec_dis−1 & chi_vec_x1−1);
p_dis_xre1_x1e1_h=length(find(chi_vec_dis(chi_vec_xre1 x1e1_ind)==1))/length(chi_vec_xre1x1e1_ind);
chi_vec_xre0x1e1_ind=find(par_vec_dis−0 & chi_vec_x1−1);
p_dis_xre0_x1e1_h=length(find(chi_vec_dis(chi_vec_xre0_x1e1_ind)==1))/length(chi_vec_xre0_x1e1_ind);
chi_vec_xre0x1e0_ind=find(par_vec_dis-0 & chi_vec_x1-0);
p_dis_xre0_x1e0_h=length(find(chi_vec_dis(chi_vec_xre0_x1e0_ind)==1))/length(chi_vec_xre0_x1e0_ind);
chi_vec_xre1 x1e0_ind=find(par_vec_dis−1 & chi_vec_x1−0);
p_dis_xre1_x1e0_h=length(find(chi_vec_dis(chi_vec_xre1_x1e0_ind)==1))/length(chi_vec_xre1_x1e0 ind);
% computing P(D/XrX2) for all states
chi_vec_xre1_x2e1 ind=find(par_vec_dis−1 & chi_vec_x2==1);
p_dis_xre1_x2e1 h=length(find(chi_vec_dis(chi_vec_xre1_x2e1 ind)==1))/length(chi_vec_xre1_x2e1 ind);
chi_vec_xre0_x2e1 ind=find(par_vec_dis−0 & chi_vec_x2==1);
p_dis_xre0_x2e1 h=length(find(chi_vec_dis(chi_vec_xre0_x2e1 ind)==1))/length(chi_vec_xre0_x2e1 ind);
chi_vec_xre0_x2e0 ind=find(par_vec_dis−0 & chi_vec_x2==0);
p_dis_xre0_x2e0 h=length(find(chi_vec_dis(chi_vec_xre0_x2e0 ind)==1))/length(chi_vec_xre0_x2e0 ind);
chi_vec_xre1_x2e0 ind=find(par_vec_dis−1 & chi_vec_x2==0);
p_dis_xre1_x2e0 h=length(find(chi_vec_dis(chi_vec_xre1_x2e0 ind)==1))/length(chi_vec_xre1_x2e0 ind);
% computing P(D/XrX3) for all states
chi_vec_xre1_x3e1 ind=find(par_vec_dis-1 & chi_vec_x3==1);
p_dis_xre1_x3e1 h=length(find(chi_vec_dis(chi_vec_xre1_x3e1 ind)==1))/length(chi_vec_xre1_x3e1 ind);
chi_vec_xre0_x3e1 ind=find(par_vec_dis-0 & chi_vec_x3==1);
p_dis_xre0_x3e1 h=length(find(chi_vec_dis(chi_vec_xre0_x3e1 ind)==1))/length(chi_vec_xre0_x3e1 ind);
chi_vec_xre0_x3e0 ind=find(par_vec_dis-0 & chi_vec_x3==0);
p_dis_xre0_x3e0 h=length(find(chi_vec_dis(chi_vec_xre0_x3e0 ind)==1))/length(chi_vec_xre0_x3e0 ind);
chi_vec_xre1_x3e0 ind=find(par_vec_dis-1 & chi_vec_x3==0);
p_dis_xre1_x3e0 h=length(find(chi_vec_dis(chi_vec_xre1_x3e0 ind)==1))/length(chi_vec_xre1_x3e0 ind);
% prediction on the training data
% could also implement this on separate test data
% computing P(D/XrX1)
p_dis_xr_x1_h=zeros(n,1);
p_dis_xr_x1_h(chi_vec_xre1_x1e1_ind)=p_dis_xre1_x1e1_h;
p_dis_xr_x1_h(chi_vec_xre0_x1e1_ind)=p_dis_xre0_x1e1_h;
p_dis_xr_x1_h(chi_vec_xre0_x1e0 ind)=p_dis_xre0_x1e0_h;
p_dis_xr_x1_h(chi_vec_xre1_x1e0_ind)=p_dis_xre1_x1e0_h;
% computing P(D/XrX2)
p_dis_xr_x2_h=zeros(n,1);
p_dis_xr_x2_h(chi_vec_xre1_x2e1 ind)=p_dis_xre1_x2e1 h;
p_dis_xr_x2_h(chi_vec_xre0_x2e1 ind)=p_dis_xre0_x2e1 h;
p_dis_xr_x2_h(chi_vec_xre0_x2e0 ind)=p_dis_xre0_x2e0 h;
p_dis_xr_x2_h(chi_vec_xre1_x2e0 ind)=p_dis_xre1_x2e0 h;
% computing P(D/XrX3)
p_dis_xr_x3_h=zeros(n,1);
p_dis_xr_x3_h(chi_vec_xre1_x3e1 ind)=p_dis_xre1_x3e1 h;
p_dis_xr_x3_h(chi_vec_xre0_x3e1 ind)=p_dis_xre0_x3e1 h;
p_dis_xr_x3_h(chi_vec_xre0_x3e0 ind)=p_dis_xre0_x3e0 h;
p_dis_xr_x3_h(chi_vec_xre1_x3e0 ind)=p_dis_xre1_x3e0 h;
%%% computing key results
% prediction using xr, x1 and x2
p_dis_xrx1x2_h=p_dis_xr_h*(p_dis_xr_x1_h/p_dis_xr_h).*(p_dis_xr_x2_h/p_dis_xr_h);
% prediction using xr, x1 and x3
p_dis_xrx1x3_h=p_dis_xr_h*(p_dis_xr_x1_h/p_dis_xr_h).*(p_dis_xr_x3_h/p_dis_xr_h);
% prediction using xr, x1, x2 and x3
p_dis_xrx1x2x3_h=p_dis_xr_h*(p_dis_xr_x1_h/p_dis_xr_h).*(p_dis_xr_x2_h/p_dis_xr_h).*(p_dis_xr_x3_h/p d is xr_h);
%%% plotting key results
%% raw data
disp_vec=[1:10000];
% figure; plot(chi_vec_dis(disp_vec),‘b.’); hold on; plot(chi_vec_dis(disp_vec),‘b’);
%% prediction using xr, x1
% plot(p_dis_xr_x1_h(disp_vec),‘gx’);
% prediction using x1
% plot(p_dis_x1_h(disp_vec),‘ro’);
%% prediction using x1 and x2%
plot(p_dis_x1x2_h(disp_vec),‘ro’);
% prediction using xr, x1 and x2%
plot(p_dis_xrx1x2_h(disp_vec),‘gx’);
%% histograms using x1, x2 (and xr)
figure; hold on;
[t1,c1]=hist(chi_vec_dis); bar(c1, log 10(t1),‘b’);
[t2,c2]=hist(p_dis_xrx1x2_h); bar(c2, log 10(t2),‘g’);
[t3,c3]=hist(p_dis_x1x2_h); bar(c3, log 10(t3),‘r’);
legend(‘Truth’, ‘Estimate of P(D|XrX1X2)’, ‘Estimate of P(D|X1X2)’);
ylabel(‘log 10(count)’);
xlabel(‘probability estimate’);
title(‘histogram of estimates P(D|X1X2), P(D|XrX1X2)’);
grid;
%% prediction using x1 and x3%
plot(p_dis_x1x3_h,‘ro’);
% prediction using xr, x1 and x3%
plot(p_dis_xrx1x3_h,‘gx’);
% histograms using x1, x3 (and xr)
figure; hold on;
[tmp3,c3]=hist(p_dis_x1x3_h); bar(c3, log 10(tmp3),‘r’);
[tmp1,c1]=hist(chi_vec_dis); bar(c1, log 10(tmp1),‘b’);
[tmp2,c2]=hist(p_dis_xrx1x3_h); bar(c2, log 10(tmp2),‘g’);
legend(‘Estimate of P(131X1X3)’, ‘Truth’, ‘Estimate of P(D|XrX1X3)’);
ylabel(‘log 10(count)’);
xlabel(‘probability estimate’);
title(‘histogram of estimates P(D|X1X3), P(D|XrX1X3)’);
grid;
%% prediction using x1, x2 and x3%
plot(p_dis_x1x2x3_h,‘ro’);
% prediction using xr, x1, x2 and x3%
plot(p_dis_xrx1x2x3_h,‘gx’);
% histograms using x1, x2, x3 (and xr)
figure; hold on;
[tm3,c3]=hist(p_dis_x1x2x3_h); bar(c3, log 10(tm3),‘r’);
[tm2,c2]=hist(p_dis_xrx1x2x3_h); bar(c2, log 10(tm2),‘g’);
[tm1,c1]=hist(chi_vec_dis); bar(c1, log 10(tm1),‘g’);
legend(‘Estimate of P(D|X1X2X3)’,‘Estimate of P(D|XrX1X2X3)’,‘Truth’);
ylabel(‘log 10(count)’);
xlabel(‘probability estimate’);
title(‘histogram of estimates P(D|X1X2X3), P(D|XrX1X2X3)’);
grid;
%%% comparing RMSE accuracy of results
% prediction using x1 (and xr)
p_dis_xr_x1_h_e=p_dis_xr_x1_h−chi_vec_dis;
p_dis_x1_h_e=p_dis_x1_h−chi_vec_dis;
p_dis_xr_x1_h_RMSE=sqrt(p_dis_xr_x1_h_e′*p_dis_xrx1_h_e/n)
p_dis_x1_h_RMSE=sqrt(p_dis_x1_h_e′*p_dis_x1_h_e/n)
% prediction using x1 and x2 (and xr)
p_dis_xrx1x2_h_e=p_dis_xrx1x2_h−chi_vec_dis;
p_dis_x1x2_h_e=p_dis_x1x2_h−chi_vec_dis;
p_dis_xrx1x2_h_RMSE=sqrt(p_dis_xrx1x2_h_e′*p_dis_xrx1x2_h_e/n)
p_dis_x1x2_h_RMSE=sqrt(p_dis_x1x2_h_e′*p_dis_x1x2_h_e/n)
% prediction using x1, x3 (and xr)
p_dis_xrx1x3_h_e=p_dis_xrx1x3_h−chi_vec_dis;
p_dis_x1x3_h_e=p_dis_x1x3_h−chi_vec_dis;
p_dis_xrx1x3_h_RMSE=sqrt(p_dis_xrx1x3_h_e′*p_dis_xrx1x3_h_e/n)
p_dis_x1x3_h_RMSE=sqrt(p_dis_x1x3_h_e′*p_dis_x1x3_h_e/n)
% prediction using x1, x2, x3 (and xr)
p_dis_xrx1x2x3_h_e=p_dis_xrx1x2x3_h−chi_vec_dis;
p_dis_x1x2x3_h_e=p_dis_x1x2x3_h−chi_vec_dis;
p_dis_xrx1x2x3_h_RMSE=sqrt(p_dis_xrx1x2x3_h_e′*p_dis_xrx1x2x3_h_e/n)
p_dis_x1x2x3_h_RMSE=sqrt(p_dis_x1x2x3_h_e′*p_dis_x1x2x3_h_e/n)

The invention claimed is:

1. A method for outputting a non-Mendelian phenotypic risk score, the method comprising:
receiving, from a first dataset, (i) genotype data for a subject having one or more non-Mendelian genes of interest and (ii) genotype data and phenotype data for one or more blood relatives of the subject that have one or more of the genes of interest,
receiving, from a second dataset, genotype population data and phenotype population data, wherein the population comprises one or more sets of two or more blood relatives,
training a model on the first and second datasets to determine a risk in the subject associated with one or more of the non-Mendelian genes of interest, and
outputting a phenotypic risk score for the subject.

2. The method of claim 1, wherein the second dataset comprises genotype population data and phenotype population data for more than one set of two or more blood relatives.

3. The method of claim 1 or 2, wherein the blood relative in the first dataset comprises one or more of the subject's mother, father, brother, sister, son, daughter, grandfather, grandmother, aunt, uncle, niece, nephew, and first cousin, and
wherein the second dataset includes two or more subjects having the same blood relationship as the subjects in the first dataset.

4. The method of any one of claims 1-3, wherein one or more of the blood relatives is a male relative.

5. The method of any one of claims 1-3, wherein one or more of the blood relatives is a female relative.

6. The method of any one of claims 1-5, wherein the first dataset includes data for more than one blood relative of the subject.

7. The method of any one of claims 1-6, wherein one or more of the blood relatives is a male relative and one or more of the blood relatives is a female relative.

8. The method of any one of claims 1-7, wherein the gene of interest is a genetic variant of interest.

9. The method of any one of claims 1-8, wherein the first dataset and second dataset include data associated with the age of onset of a phenotype.

10. A system comprising:
a processor,
a memory coupled to the processor to store instructions which, when executed by the processor, cause the processor to perform operations, the operations including:
receiving, from a first dataset, (i) genotype data for a subject having one or more non-Mendelian genes of interest and (ii) genotype data and phenotype data for one or more blood relatives of the subject that have one or more of the genes of interest,
receiving, from a second dataset, genotype population data and phenotype population data, wherein the population comprises one or more sets of two or more blood relatives,
training a model on the first and second datasets to determine a risk in the subject associated with one or more of the non-Mendelian genes of interest, and
outputting a phenotypic risk score for the subject.

11. A non-transitory machine-readable medium having instructions stored therein which, when executed by a processor, cause the processor to perform operations, the operations comprising:
receiving, from a first dataset, (i) genotype data for a subject having one or more non-Mendelian genes of interest and (ii) genotype data and phenotype data for one or more blood relatives of the subject that have one or more of the genes of interest,
receiving, from a second dataset, genotype data and phenotype population data, wherein the population comprises one or more sets of two or more blood relatives,
training, by the processor, a model on the first and second datasets to determine a genetic risk in the subject associated with one or more of the non-Mendelian genes of interest, and
outputting a phenotypic risk score for the subject.

12. The non-transitory machine-readable medium of claim 11, wherein the second dataset comprises genotype population data and phenotype population data for more than one set of two or more blood relatives.

13. The non-transitory machine-readable medium of claim 11 or 12, wherein the blood relative in the first dataset comprises one or more of the subject's mother, father, brother, sister, son, daughter, grandfather, grandmother, aunt, uncle, niece, nephew, and first cousin, and
wherein the second dataset includes two or more subjects having the same blood relationship as the subjects in the first dataset.

14. The non-transitory machine-readable medium of any one of claims 11-13, wherein one or more of the blood relatives is a male relative.

15. The non-transitory machine-readable medium of any one of claims 11-13, wherein one or more of the blood relatives is a female relative.

16. The non-transitory machine-readable medium of any one of claims 11-15, wherein the first dataset includes data for more than one blood relative of the subject.

17. The non-transitory machine-readable medium of any one of claims 11-16, wherein one or more of the blood relatives is a male relative and one or more of the relatives is a female relative.

18. The non-transitory machine-readable medium of any one of claims 11-17, wherein the gene of interest is a genetic variant of interest.

19. The non-transitory machine-readable medium of any one of claims 11-18, wherein the first dataset and second dataset include data associated with the age of onset of a phenotype.

20. A method for outputting a polygenic risk score, the method comprising:
receiving, from a first dataset, (i) genotype data for a subject having one or more non-Mendelian genes of interest and (ii) genotype data and phenotype data for one or more blood relatives of the subject that have one or more of the non-Mendelian genes of interest,
receiving, from a second dataset, genotype population data and phenotype population data, wherein the population comprises one or more sets of two or more blood relatives,
training a model on the first and second datasets to predict a risk in the subject based on the one or more non-Mendelian genes of interest, and
outputting a polygenic risk score for the subject.

21. The method of claim 20, the method comprising:
training a model on the first and second datasets to predict how the risk in the subject is modified by one or more non-Mendelian genes of interest, relative to the risk in the subject given the phenotype data of the blood relatives.

22. The method of any one of claims 1-21, further comprising treating the subject based on the risk score.
